(19) United States
(12) Patent Application Publication
(10) Pub. No.: US 2023/0044209 A1
(43) Pub. Date: Feb. 9, 2023

(54) METHOD AND SYSTEM FOR DETECTING AND CLASSIFYING SEGMENTS OF SIGNALS FROM EEG-RECORDINGS

(71) Applicant: PROLIRA B.V., Utrecht (NL)

(72) Inventors: Philip Michael ZEMAN, Utrecht (NL); Rutger Olof VAN MERKERK, Utrecht (NL); Arnout Tim VAN ZON, Utrecht (NL)

(21) Appl. No.: 17/793,108

(22) PCT Filed: Nov. 20, 2020
(86) PCT No.: PCT/EP2020/082823

§ 371 Date: Jul. 15, 2022

(30) Foreign Application Priority Data
Jan. 16, 2020 (EP) 20152275.2

(51) Int. Cl. A61B 5/00; A61B 5/369

(52) U.S. Cl. CPC A61B 5/0006; A61B 5/369

(57) ABSTRACT

A data processing method for detecting and classifying a segment of a signal that is obtained from a single-channel EEG-recording as a target signal segment or as a non-target signal segment. The method includes a voting process to determine whether classification of a first detected segment of the signal as a target signal segment or classification of a second detected segment of the signal as a non-target signal segment is correct. A device and a system that are configured and arranged to perform the data processing method.

### FIELD OF THE INVENTION

The present invention relates to a method for detecting and classifying a segment of a signal that is obtained from a single-channel EEG-recording as a target signal segment or as a non-target signal segment. The invention further relates to a device and a system that are configured and arranged to perform the method according to the invention.

### BACKGROUND OF THE INVENTION

Delirium is an acute disturbance of consciousness and cognition that usually fluctuates over time. It is a common disorder, with reported incidences of more than 60% during Intensive Care Unit (ICU) stay and over 15% on a geriatric ward or medium care unit. Delirium is associated with higher mortality, longer hospitalization, long-term cognitive impairment and increased costs. There are three different subtypes of delirium based on psychomotor behavior, i.e. hypoactive, hyperactive and mixed-type delirium.

Despite its frequency and impact, recognition of delirium by health care professionals is poor. Exceptions are hyperactive forms of delirium, but these are relatively rare. Furthermore, delayed treatment of delirium in ICU patients was found to increase mortality. To improve early diagnosis and treatment, the Society of Critical Care Medicine and the American Psychiatric Association recommend daily monitoring of delirium in ICU patients.

Various delirium assessment tools have been developed including for example the Confusion Assessment Method for the ICU (CAM-ICU) as well as methods and systems that involve electroencephalography (EEG) using for example single-channel EEG-recordings.

The disclosure by T. Numan et al.: “Delirium detection using relative delta power based on 1-minute single-channel EEG: a multicentre study”, British Journal of Anaesthesia, vol. 122, no. 1, 1 Jan. 2019, pages 60-68 discloses delirium detection using an algorithm for EEG analyses based on spectral analysis. The algorithm provides a normalized delta power, the so-called relative delta power, by dividing the power in the so-called delta EEG frequency band (1-4 Hz) and in the frequency band of 1-6 Hz by the power in the total EEG frequency band of 1-30 Hz.

A disadvantage of known delirium assessment methods and systems that use single-channel EEG-recordings is that they do not provide a reliable distinction between target signal segments and non-target signal segments of a single-channel EEG-recording, in particular when the target signal segments and the non-target signal segments of the single-channel EEG-recording look alike. It is noted that in the context of the present invention target signal segments of a single-channel EEG-recording are to be construed as signal segments that are, for example, indicative for a patient being delirious or suffering from related encephalopathy, whereas non-target signal segments of a single-channel EEG-recording are to be construed as signal segments that are indicative for non-target brain signals or artifacts such as for example eye artifacts, artifacts related to muscle activity, or artifacts related to a combination of such artifacts.

In view of the abovementioned disadvantage of known delirium assessment methods and systems using single-channel EEG-recordings, there is a need to provide a method and a system that enables improved distinction between target signal segments and non-target signal segments of a single-channel EEG-recording.

### SUMMARY OF THE INVENTION

It is an object of the present invention to provide a method for detecting and classifying a segment of a signal that is obtained from a single-channel EEG-recording as a target signal segment or as a non-target signal segment that pre-empts or at least reduces the abovementioned disadvantage and/or other disadvantages associated with known delirium assessment methods using single-channel EEG-recordings.

It is also an object of the present invention to provide a device and a system for performing the method according to the invention.

Aspects of the present invention are set out in the accompanying independent and dependent claims. Features from the dependent claims may be combined with features from the independent claim as appropriate and not merely as explicitly set out in the claims.

At least one of the abovementioned objects is achieved by a data processing method for detecting and classifying a segment of a signal that is obtained from a single-channel EEG-recording as a target signal segment or as a non-target signal segment, the method comprising:
providing a signal that is obtained from a single-channel EEG-recording;
applying to said signal a target parameter set, which is indicative for a plurality of reference target signal segments that are obtained from reference single-channel EEG-recordings, to detect a first signal segment of said signal and to classify the detected first signal segment as a target signal segment, wherein the target parameter set comprises wavelet coefficients that are determined using wavelet decomposition of the plurality of reference target signal segments;
assigning a first time stamp to the detected first signal segment;
applying to said signal a non-target parameter set, which is indicative for a plurality of reference non-target signal segments that are obtained from reference single-channel EEG-recordings, to detect a second signal segment of said signal and to classify the detected second signal segment as a non-target signal segment, wherein the non-target parameter set comprises wavelet coefficients that are determined using wavelet decomposition of the plurality of reference non-target signal segments;
assigning a second time stamp to the detected second signal segment;
determining a temporal proximity of the first time stamp and the second time stamp;
based on said determined temporal proximity, determining if a voting process is required to determine whether classification of the detected first signal segment as a target signal segment or classification of the detected second signal segment as a non-target signal segment is correct; and
upon establishing that said voting process is required, performing said voting process.

In this way, an improved distinction between target signal segments and non-target signal segments of a signal that is obtained from a single-channel EEG-recording can be made and therefore false positive detections or incorrect classifications for that matter can be reduced.

The plurality of reference target signal segments and the plurality of reference non-target signal segments can be obtained from signals that are obtained from single-channel EEG-recordings. The person skilled in the art will appreciate that single-channel EEG-recordings can be acquired in various known ways including for example using a differential electrode pair, using a single electrode in combination with a reference (REF) electrode or a ground (GND) electrode, or using a differential electrode pair in combination with a REF electrode or a GND electrode. In accordance with the latter exemplary electrode configuration, the individual electrodes of the differential electrode pair can be positioned at specific midline frontal, e.g. Fz, midline vertex, e.g. Cz, midline parietal, e.g. Pz, left medial temporal, e.g. T3, right medial temporal, e.g. T4, positions on a patient's scalp in accordance with an extended 10-20 EEG system with the REF electrode positioned, for example, on an ear of the patient.

In addition to the above-mentioned examples for acquiring single-channel EEG-recordings, the person skilled in the art will appreciate that a single-channel EEG-recording in the context of the present invention can also be a single channel from a standard 10-20 EEG montage or from any other multichannel EEG montage for that matter. Regarding the latter interpretation of the phrase “single-channel EEG-recording”, it is noted that in the context of the present invention, the plurality of reference target signal segments and the plurality of reference non-target signal segments are obtained exclusively from data of a single channel.

The single-channel EEG-recordings used to obtain the plurality of reference target signal segments and the plurality of reference non-target signal segments can have a predefined duration of for example 15 minutes. However, the person skilled in the art will appreciate that any suitable predefined duration can be used as long as the acquired single-channel EEG-recordings enable obtaining suitable reference target signal segments and reference non-target signal segments.

The reference target signal segments of the plurality of reference target signal segments can be mutually different. The same holds for the reference non-target signal segments of the plurality of non-target signal segments. The plurality of reference target signal segments can for example comprise more than 1000 reference target signal segments. The same holds for the plurality of non-target signal segments.

The target parameter set that is indicative for the plurality of reference target signal segments can be determined by processing and analyzing the reference target signal segments of the plurality of reference target signal segments. The aforementioned processing and analyzing can be done using a training process that involves a machine learning algorithm that can for example use deep neural networks. The processing and analyzing can for example be done in the frequency domain. The target parameter set can be construed as an aggregate parameter set as it comprises parameters that are indicative for the plurality of reference target signal segments.

Upon determining the target parameter set, it can be used to detect a first signal segment of a single-channel EEG-recording that is acquired in one of the above-mentioned ways and to classify the detected first signal segment as a target signal segment, i.e. a signal segment that is indicative for a patient being delirious or suffering from related encephalopathy. Upon detecting the first signal segment, it can be marked by assigning the first time stamp to it.

In an analogous way, the non-target parameter set that is indicative for the plurality of reference non-target signal segments can be determined by processing and analyzing the reference non-target signal segments of the plurality of reference non-target signal segments. The aforementioned processing and analyzing can be done using another training process that involves another machine learning algorithm that can for example use deep neural networks. The processing and analyzing can for example be done in the frequency domain. The non-target parameter set can be construed as an aggregate parameter set as it comprises parameters that are indicative for the plurality of reference non-target signal segments.

Upon determining the non-target parameter set, it can be used to detect a second signal segment of the same signal that was obtained from the single-channel EEG-recording that is acquired in one of the above-mentioned ways and to classify the detected second signal segment as a non-target signal segment, i.e. a signal segment that is indicative for artifacts such as for example eye artifacts, artifacts related to muscle activity, or artifacts related to a combination of such artifacts. Upon detecting the second signal segment, it can be marked by assigning the second time stamp to it.

In accordance with the present invention, determining a temporal proximity of the first time stamp and the second time stamp can be done in several different ways.

A first exemplary way of doing this involves dividing the obtained first signal in time intervals of a predefined length. Preferably, the time intervals have a predefined equal length. These time intervals can be referred to as bins. Preferably, the time intervals or bins have a predefined equal length. The predefined length of the time intervals can be chosen depending on specific requirements such as desired accuracy. Suitable predefined lengths of the time intervals range between 0.25 s and 3 s. Preferably, the time intervals have a predefined length of 1 s. Determining the temporal proximity of the first time stamp and the second time stamp is based on establishing if the first time stamp and the second time stamp fall within the same time interval or not. If the first time stamp and the second time stamp fall within the same time interval or bin, then the voting process is required and will be performed to determine whether classification of the detected first signal segment as a target signal segment or classification of the detected second signal segment as a non-target signal segment is correct. If the first time stamp and the second time stamp fall in different time intervals, i.e. the first time stamp and the second time stamp do not fall within the same bin, then the voting process is not required and preferably the voting process is not performed. In this case, the classification of the detected first signal segment as being a target signal segment and the classification of the detected second signal segment as being a non-target signal segment are most likely both correct.

A second way of determining the temporal proximity of the first time stamp and the second time stamp involves determining a time difference between the first time stamp and the second time stamp. The determined time difference is compared with a predefined threshold. The person skilled in the art will appreciate that the predefined threshold is chosen such that when the determined time difference between the first time stamp and the second time stamp is smaller than the threshold, it can be not likely or not possible at all that the classification of the detected first signal segment as being a target signal segment and the classification of the detected second signal segment as being a non-target signal segment can both be correct. Hence, when the determined time difference is smaller than the predefined threshold the voting process is required to determine whether classification of the detected first signal segment as a target signal segment or the classification of the detected second signal segment as a non-target signal segment is correct. If the determined time difference is equal to or larger than the threshold then the classification of the detected first signal segment as being a target signal segment and the classification of the detected second signal segment as being a non-target signal segment are most likely both correct.

The person skilled in the art will appreciate that any suitable predefined threshold can be chosen as long as it allows to establish whether the classification of the detected first signal segment as being a target signal segment and/or the classification of the detected second signal segment as being a non-target signal segment can be correct. Suitable values for the predefined threshold range between 0.25 s and 3 s. Preferably, the threshold is 1 s.

Based on the above, it will be clear that the voting process eliminates one of the classifications. As a result, the method according to the present invention can reduce false positive detections or incorrect classifications for that matter.

Based on the above, an example of the data processing method according to the present invention is a method for detecting and classifying a segment of a signal that is obtained from a single-channel EEG-recording as a target signal segment or as a non-target signal segment, the method comprising:
providing a signal that is obtained from a single-channel EEG-recording;
applying to said signal a target parameter set that is indicative for a plurality of reference target signal segments that are obtained from reference single-channel EEG-recordings to detect a first signal segment and to classify the detected first signal segment as a target signal segment;
assigning a first time stamp to the detected first signal segment;
applying to said signal a non-target parameter set that is indicative for a plurality of reference non-target signal segments that are obtained from reference single-channel EEG-recordings to detect a second signal segment and to classify the detected second signal segment as a non-target signal segment;
assigning a second time stamp to the detected second signal segment;
determining a temporal proximity of the first time stamp and the second time stamp;
based on said determined temporal proximity, determining if a voting process is required to determine whether classification of the detected first signal segment as a target signal segment or classification of the detected second signal segment as a non-target signal segment is correct; and
upon establishing that said voting process is required, performing said voting process.

In an embodiment of the method according to the invention, performing the voting process comprises:
generating a first signal sample that comprises the detected first signal segment;
matching the first signal sample with the plurality of reference target signal segments to determine a best target match;
generating a second signal sample that comprises the detected second signal segment;
matching the second signal sample with the plurality of reference non-target signal segments to determine a best non-target match;
applying metrics to the first signal sample, the best target match, the second signal sample and the best non-target match to determine:
   whether the classification of the detected first signal segment as a target signal segment is correct; or
   whether the classification of the detected second signal segment as a non-target signal segment is correct.

In accordance with the present invention, matching the first signal sample with the plurality of reference target signal segments to determine the best target match can involve for example curve fitting in the time domain of the first signal sample with the plurality of reference target signal segments. In an analogous way, matching the second signal sample with the plurality of reference non-target signal segments to determine the best non-target match can involve for example curve fitting in the time domain of the second signal sample with the plurality of reference non-target signal segments.

In accordance with the present invention, curve fitting in the time domain may include comparing the signal shape of the first signal sample with the signal shapes of the reference target signal segments of the plurality of reference target signal segments and comparing of the signal shape of the second signal sample with the signal shapes of the reference non-target signal segments of the plurality of reference non-target signal segments. In this case the curve fit resulting in for example the smallest residue can be chosen to determine the best target match and the best non-target match, respectively. However, other aspects related to the curve fitting process can of course also be regarded to determine the best target match and the best non-target match, respectively.

The person skilled in the art will appreciate that curve fitting in the time domain is just an example of the analysis methods that are available to determine the best target match and the best non-target match, respectively. Examples of analysis methods include for example Fast Fourier Transform (FFT), linear signal analysis techniques involving determination of coherence, non-linear signal analysis techniques involving determination of phase synchronization and/or generalized synchronization, template matching, and parametric models including the use of wavelets.

In accordance with the present invention, applying metrics to the first signal sample, the best target match, the second signal sample and the best non-target match can be done in several different ways. A first way of doing this is by establishing and comparing a correlation in the time domain. A second way of doing this is by establishing and comparing a goodness of fit in the wavelet domain. By applying either one of these techniques it can be determined whether the classification of the detected first signal segment as being a target signal segment or the classification of the detected second signal segment as being a non-target signal segment is correct.

As a result of the above, it will be clear that the voting process can eliminate one of the two classifications and thereby will determine the final classification as target signal segment or as non-target signal segment. As a result, false positive classifications or incorrect detections for that matter can be reduced.

In an embodiment of the method according to the invention, performing the voting process comprises:
generating a first signal sample that comprises the detected first signal segment;
matching the first signal sample with a set of reference target signal segments that is based on the plurality of reference target signal segments to determine a best target match;
generating a second signal sample that comprises the detected second signal segment;
matching the second signal sample with a set of reference non-target signal segments that is based on the plurality of reference non-target signal segments to determine a best non-target match;
applying metrics to the first signal sample, the best target match, the second signal sample and the best non-target match to determine:
   whether the classification of the detected first signal segment as a target signal segment is correct; or
   whether the classification of the detected second signal segment as a non-target signal segment is correct.

The person skilled in the art will appreciate that the same considerations as mentioned above regarding the previous embodiment of the present invention equally apply to the steps of matching the first signal sample and the second signal sample with the set of reference target signal segments that is based on the plurality of reference target signal samples, and the set of reference non-target signal segments that is based on the plurality of reference non-target signal samples of the currently mentioned embodiment of the present invention.

Moreover, the person skilled in the art will appreciate that the same considerations as mentioned above regarding the previous embodiment of the present invention equally apply to the step of applying metrics to the first signal sample, the best target match, the second signal sample and the best non-target match of the currently mentioned embodiment of the present invention.

Furthermore, it will be clear that by using a set of reference target signal segments that is based on the plurality of reference target signal segments and a set of reference non-target signal segments that is based on the plurality of reference non-target signal segments the voting process can eliminate one of the two classifications without having to use all reference target signal segments of the plurality of reference target signal segments and all reference non-target signal segments of the plurality of reference non-target signal segments, respectively. As a result, the voting process can be performed faster.

In an embodiment of the method according to the invention, the method further comprises removing the classification of the detected first signal segment or the classification of the detected second signal segment that based on the voting process is incorrect. The person skilled in the art will appreciate that the voting process of the method according to the present invention results in a so-called winner, i.e. either the classification of the detected first signal segment as being a target signal segment or the classification of the detected second signal segment as being a non-target signal segment is correct. The classification that in accordance with the voting process is to be regarded as incorrect will be removed.

In an embodiment of the method according to the invention, a predetermined detection boundary, which is determined based on the target parameter set and/or the non-target parameter set, is applied that allows classification of detected signal segments as target signal segments or as non-target signal segments. The detection boundary can for example be a boundary plane in the feature space of wavelet coefficients that is determined by training on a labeled training set of target signal segments and non-target signal segments. In this way, another way to make a distinction between target signal segments and non-target signal segments of a single-channel EEG-recording can be provided. Hence, false positive detections or incorrect classifications for that matter can be reduced.

In an example of the method according to the invention, based on the target parameter set and/or the non-target parameter set a detection boundary is determined that allows classification of detected signal segments as target signal segments or as non-target signal segments.

In an embodiment of the method according to the invention, the target parameter set comprises wavelet coefficients that are determined using wavelet decomposition of the plurality of reference target signal segments, and the non-target parameter set comprises wavelet coefficients that are determined using wavelet decomposition of the plurality of reference non-target signal segments.

The wavelet coefficients can be determined by wavelet decomposition of the reference target signal segments of the plurality of reference target signal segments and of the reference non-target signal segments of the plurality of the reference non-target signal segments. Upon determining the respective wavelet coefficients for the reference target signal segments and for the reference non-target signal segments, a training process that involves a machine learning algorithm can be used to identify the wavelet coefficients that are most representative for the reference target signal segments and for the reference non-target signal segments, respectively. The machine learning algorithm can for example use deep neural networks. The person skilled in the art will appreciate that the target parameter set preferably comprises the wavelet coefficients that are most representative for the reference target signal segments and that the non-target parameter set preferably comprises the wavelet coefficients that are most representative for the reference non-target signal segments. The person skilled in the art will appreciate that the thus obtained target parameter set and the non-target parameter set can comprise statistical average values of the wavelet coefficients that are most representative for the plurality of reference target signal segments and for the plurality of reference non-target signal segments, respectively.

In an embodiment of the method according to the invention, the method further comprises determining an optimized target parameter set that comprises wavelet coefficients that are indicative specifically for the plurality of reference target signal segments and/or an optimized non-target parameter set that comprises wavelet coefficients that are indicative specifically for the plurality of reference non-target signal segments.

Determination of the optimized target parameter set and the optimized non-target parameter set can be achieved in several ways. A first way of doing this involves comparing of the wavelet coefficients of the target parameter set and the wavelet coefficients of the non-target parameter set, wherein wavelet coefficients that occur both in the target parameter set and in the non-target parameter set are removed from the target parameter set and/or from the non-target parameter set. In this way, overlap between the target parameter set and the non-target parameter set can be reduced.

A second way of determining the optimized target parameter set and the optimized non-target parameter set involves using the resulting sensitivity and specificity of a system, e.g. a classifier unit, that is adapted to classify a detected first signal segment as being a target signal segment and a detected second signal segment as being a non-target signal segment.

As a result of any one of the above-mentioned first way and second way, the thus optimized target parameter set and non-target parameter set enable an improved distinction between target signal segments and non-target signal segments of a single-channel EEG-recording. Hence, false positive detections or incorrect classifications for that matter can be reduced.

In an embodiment of the method according to the invention, based on the optimized target parameter set and/or the optimized non-target parameter set a detection boundary is determined that allows improved classification of detected signal segments as target signal segments or as non-target signal segments. In this way, an improved distinction between target signal segments and non-target signal segments of a single-channel EEG-recording can be achieved. Hence, false positive detections or incorrect classifications for that matter can be reduced.

According to another aspect of the present invention, a device is provided that is configured and arranged to be used with a system that is configured and arranged to detect and classify a segment of a signal that is obtained from a single-channel EEG-recording as a target signal segment or as a non-target signal segment, the device having a database comprising at least one of:
a plurality of reference target signal segments that are obtained from reference single-channel EEG-recordings;
a set of reference target signal segments that is based on the plurality of reference target signal segments;

a plurality of reference non-target signal segments that are obtained from reference single-channel EEG-recordings;

a set of reference non-target signal segments that is based on the plurality of reference non-target signal segments;

a target parameter set that is indicative for the plurality of reference target signal segments; and

a non-target parameter set that is indicative for a plurality of reference non-target signal segments.

The device can be construed as a detector that comprises dedicated parameter sets, i.e. the target parameter set that is indicative for the plurality of reference target signal segments and the non-target parameter set that is indicative for the plurality of reference non-target signal segments, wherein the dedicated parameter sets can be obtained via respective training processes as described above that may involve respective machine learning algorithms that may use respective deep neural networks.

The device according to the invention enables an improved distinction between target signal segments and non-target signal segments of a signal that is obtained from a single-channel EEG-recording. As a result, false positive detections or incorrect classifications for that matter as discussed above can be reduced.

According to yet another aspect of the present invention, a system is provided that is configured and arranged to detect and classify a segment of a signal that is obtained from a single-channel EEG-recording as a target signal segment or as a non-target signal segment, the system comprising a processor that is configured and arranged to perform the method according to the present invention on said signal when being operatively connected to the device according to the present invention.

In this way, the system and the device when being operatively connected can be used to achieve an improved distinction between target signal segments and non-target signal segments of a signal that is obtained from a single-channel EEG-recording. As a result, false positive detections or incorrect classifications for that matter as discussed above can be reduced. The person skilled in the art will appreciate that the device and the system can be implemented as separate units. However, the device and the system can also be implemented as an integrated unit.

According to a further aspect of the present invention, a system is provided that is configured and arranged to detect and classify a segment of a signal that is obtained from a single-channel EEG-recording as a target signal segment or as a non-target signal segment, the system comprising a processor that is configured and arranged to perform on said signal when being operatively connected to the device according to the present invention the process steps of:
providing a signal that is obtained from a single-channel EEG-recording;
applying to said signal a target parameter set, which is indicative for a plurality of reference target signal segments that are obtained from reference single-channel EEG-recordings, to detect a first signal segment of said signal and to classify the detected first signal segment as a target signal segment, wherein the target parameter set comprises wavelet coefficients that are determined using wavelet decomposition of the plurality of reference target signal segments;
assigning a first time stamp to the detected first signal segment;
applying to said signal a non-target parameter set, which is indicative for a plurality of reference non-target signal segments that are obtained from reference single-channel EEG-recordings, to detect a second signal segment of said signal and to classify the detected second signal segment as a non-target signal segment, wherein the non-target parameter set comprises wavelet coefficients that are determined using wavelet decomposition of the plurality of reference non-target signal segments;
assigning a second time stamp to the detected second signal segment;
determining a temporal proximity of the first time stamp and the second time stamp;
based on said determined temporal proximity, determining if a voting process is required to determine whether classification of the detected first signal segment as a target signal segment or classification of the detected second signal segment as a non-target signal segment is correct; and
upon establishing that said voting process is required, performing said voting process.

In this way, an improved distinction between target signal segments and non-target signal segments of a signal that is obtained from a single-channel EEG-recording can be made and therefore false positive detections or incorrect classifications for that matter can be reduced.

The plurality of reference target signal segments and the plurality of reference non-target signal segments can be obtained from signals that are obtained from single-channel EEG-recordings. The person skilled in the art will appreciate that single-channel EEG-recordings can be acquired in various known ways including for example using a differential electrode pair, using a single electrode in combination with a reference (REF) electrode or a ground (GND) electrode, or using a differential electrode pair in combination with a REF electrode or a GND electrode. In accordance with the latter exemplary electrode configuration, the individual electrodes of the differential electrode pair can be positioned at specific midline frontal, e.g. Fz, midline vertex, e.g. Cz, midline parietal, e.g. Pz, left medial temporal, e.g. T3, right medial temporal, e.g. T4, positions on a patient's scalp in accordance with an extended 10-20 EEG system with the REF electrode positioned, for example, on an ear of the patient. The single-channel EEG-recordings used to obtain the plurality of reference target signal segments and the plurality of reference non-target signal segments can have a predefined duration of for example 15 minutes. However, the person skilled in the art will appreciate that any suitable predefined duration can be used as long as the acquired single-channel EEG-recordings enable obtaining suitable reference target signal segments and reference non-target signal segments.

The reference target signal segments of the plurality of reference target signal segments can be mutually different. The same holds for the reference non-target signal segments of the plurality of non-target signal segments. The plurality of reference target signal segments can for example comprise more than 1000 reference target signal segments. The same holds for the plurality of non-target signal segments.

The target parameter set that is indicative for the plurality of reference target signal segments can be determined by processing and analyzing the reference target signal segments of the plurality of reference target signal segments. The aforementioned processing and analyzing can be done using a training process that involves a machine learning algorithm that can for example use deep neural networks. The processing and analyzing can for example be done in the frequency domain. The target parameter set can be construed as an aggregate parameter set as it comprises parameters that are indicative for the plurality of reference target signal segments. The target parameter set comprises wavelet coefficients that are determined using wavelet decomposition of the plurality of reference target signal segments.

Upon determining the target parameter set, it can be used to detect a first signal segment of a single-channel EEG-recording that is acquired in one of the above-mentioned ways and to classify the detected first signal segment as a target signal segment, i.e. a signal segment that is indicative for a patient being delirious or suffering from related encephalopathy. Upon detecting the first signal segment, it can be marked by assigning the first time stamp to it.

In an analogous way, the non-target parameter set that is indicative for the plurality of reference non-target signal segments can be determined by processing and analyzing the reference non-target signal segments of the plurality of reference non-target signal segments. The aforementioned processing and analyzing can be done using another training process that involves another machine learning algorithm that can for example use deep neural networks. The processing and analyzing can for example be done in the frequency domain. The non-target parameter set can be construed as an aggregate parameter set as it comprises parameters that are indicative for the plurality of reference non-target signal segments. The non-target parameter set comprises wavelet coefficients that are determined using wavelet decomposition of the plurality of reference non-target signal segments.

The wavelet coefficients can be determined by wavelet decomposition of the reference target signal segments of the plurality of reference target signal segments and of the reference non-target signal segments of the plurality of the reference non-target signal segments. Upon determining the respective wavelet coefficients for the reference target signal segments and for the reference non-target signal segments, a training process that involves a machine learning algorithm can be used to identify the wavelet coefficients that are most representative for the reference target signal segments and for the reference non-target signal segments, respectively. The machine learning algorithm can for example use deep neural networks. The person skilled in the art will appreciate that the target parameter set preferably comprises the wavelet coefficients that are most representative for the reference target signal segments and that the non-target parameter set preferably comprises the wavelet coefficients that are most representative for the reference non-target signal segments. The person skilled in the art will appreciate that the thus obtained target parameter set and the non-target parameter set can comprise statistical average values of the wavelet coefficients that are most representative for the plurality of reference target signal segments and for the plurality of reference non-target signal segments, respectively.

Upon determining the non-target parameter set, it can be used to detect a second signal segment of the same signal that was obtained from the single-channel EEG-recording that is acquired in one of the above-mentioned ways and to classify the detected second signal segment as a non-target signal segment, i.e. a signal segment that is indicative for artifacts such as for example eye artifacts, artifacts related to muscle activity, or artifacts related to a combination of such artifacts. Upon detecting the second signal segment, it can be marked by assigning the second time stamp to it.

In accordance with the present invention, determining a temporal proximity of the first time stamp and the second time stamp can be done in several different ways.

A first exemplary way of doing this involves dividing the obtained first signal in time intervals of a predefined length. Preferably, the time intervals have a predefined equal length. These time intervals can be referred to as bins. Preferably, the time intervals or bins have a predefined equal length. The predefined length of the time intervals can be chosen depending on specific requirements such as desired accuracy. Suitable predefined lengths of the time intervals range between 0.25 s and 3 s. Preferably, the time intervals have a predefined length of 1 s. Determining the temporal proximity of the first time stamp and the second time stamp is based on establishing if the first time stamp and the second time stamp fall within the same time interval or not. If the first time stamp and the second time stamp fall within the same time interval or bin, then the voting process is required and will be performed to determine whether classification of the detected first signal segment as a target signal segment or classification of the detected second signal segment as a non-target signal segment is correct. If the first time stamp and the second time stamp fall in different time intervals, i.e. the first time stamp and the second time stamp do not fall within the same bin, then the voting process is not required and preferably the voting process is not performed. In this case, the classification of the detected first signal segment as being a target signal segment and the classification of the detected second signal segment as being a non-target signal segment are most likely both correct.

A second way of determining the temporal proximity of the first time stamp and the second time stamp involves determining a time difference between the first time stamp and the second time stamp. The determined time difference is compared with a predefined threshold. The person skilled in the art will appreciate that the predefined threshold is chosen such that when the determined time difference between the first time stamp and the second time stamp is smaller than the threshold, it can be not likely or not possible at all that the classification of the detected first signal segment as being a target signal segment and the classification of the detected second signal segment as being a non-target signal segment can both be correct. Hence, when the determined time difference is smaller than the predefined threshold the voting process is required to determine whether classification of the detected first signal segment as a target signal segment or the classification of the detected second signal segment as a non-target signal segment is correct. If the determined time difference is equal to or larger than the threshold then the classification of the detected first signal segment as being a target signal segment and the classification of the detected second signal segment as being a non-target signal segment are most likely both correct.

The person skilled in the art will appreciate that any suitable predefined threshold can be chosen as long as it allows to establish whether the classification of the detected first signal segment as being a target signal segment and/or the classification of the detected second signal segment as being a non-target signal segment can be correct. Suitable values for the predefined threshold range between 0.25 s and 3 s. Preferably, the threshold is 1 s.

Based on the above, it will be clear that the voting process eliminates one of the classifications. As a result, the system according to the present invention can reduce false positive detections or incorrect classifications for that matter.

In an example of the system according to the invention, a system is provided that is configured and arranged to detect and classify a segment of a signal that is obtained from a single-channel EEG-recording as a target signal segment or as a non-target signal segment, the system comprising a processor that is configured and arranged to perform the method according to the present invention on said signal when being operatively connected to the device according to the present invention.

In an embodiment of the system according to the invention, the processor is configured and arranged to perform the voting process comprising the process steps of:
generating a first signal sample that comprises the detected first signal segment;
matching the first signal sample with the plurality of reference target signal segments to determine a best target match;
generating a second signal sample that comprises the detected second signal segment;
matching the second signal sample with the plurality of reference non-target signal segments to determine a best non-target match;
applying metrics to the first signal sample, the best target match, the second signal sample and the best non-target match to determine:
   whether the classification of the detected first signal segment as a target signal segment is correct; or
   whether the classification of the detected second signal segment as a non-target signal segment is correct.

In accordance with the present invention, matching the first signal sample with the plurality of reference target signal segments to determine the best target match can involve for example curve fitting in the time domain of the first signal sample with the plurality of reference target signal segments. In an analogous way, matching the second signal sample with the plurality of reference non-target signal segments to determine the best non-target match can involve for example curve fitting in the time domain of the second signal sample with the plurality of reference non-target signal segments.

In accordance with the present invention, curve fitting in the time domain may include comparing the signal shape of the first signal sample with the signal shapes of the reference target signal segments of the plurality of reference target signal segments and comparing of the signal shape of the second signal sample with the signal shapes of the reference non-target signal segments of the plurality of reference non-target signal segments. In this case the curve fit resulting in for example the smallest residue can be chosen to determine the best target match and the best non-target match, respectively. However, other aspects related to the curve fitting process can of course also be regarded to determine the best target match and the best non-target match, respectively.

The person skilled in the art will appreciate that curve fitting in the time domain is just an example of the analysis methods that are available to determine the best target match and the best non-target match, respectively. Examples of analysis methods include for example Fast Fourier Transform (FFT), linear signal analysis techniques involving determination of coherence, non-linear signal analysis techniques involving determination of phase synchronization and/or generalized synchronization, template matching, and parametric models including the use of wavelets.

In accordance with the present invention, applying metrics to the first signal sample, the best target match, the second signal sample and the best non-target match can be done in several different ways. A first way of doing this is by establishing and comparing a correlation in the time domain. A second way of doing this is by establishing and comparing a goodness of fit in the wavelet domain. By applying either one of these techniques it can be determined whether the classification of the detected first signal segment as being a target signal segment or the classification of the detected second signal segment as being a non-target signal segment is correct.

As a result of the above, it will be clear that the voting process can eliminate one of the two classifications and thereby will determine the final classification as target signal segment or as non-target signal segment. As a result, false positive classifications or incorrect detections for that matter can be reduced.

In an embodiment of the system according to the invention, the processor is configured and arranged to perform the voting process comprising the process steps of:
generating a first signal sample that comprises the detected first signal segment;
matching the first signal sample with a set of reference target signal segments that is based on the plurality of reference target signal segments to determine a best target match;
generating a second signal sample that comprises the detected second signal segment;
matching the second signal sample with a set of reference non-target signal segments that is based on the plurality of reference non-target signal segments to determine a best non-target match;
applying metrics to the first signal sample, the best target match, the second signal sample and the best non-target match to determine:
   whether the classification of the detected first signal segment as a target signal segment is correct; or
   whether the classification of the detected second signal segment as a non-target signal segment is correct.

The person skilled in the art will appreciate that the same considerations as mentioned above regarding the previous embodiment of the present invention equally apply to the steps of matching the first signal sample and the second signal sample with the set of reference target signal segments that is based on the plurality of reference target signal samples, and the set of reference non-target signal segments that is based on the plurality of reference non-target signal samples of the currently mentioned embodiment of the present invention.

Moreover, the person skilled in the art will appreciate that the same considerations as mentioned above regarding the previous embodiment of the present invention equally apply to the step of applying metrics to the first signal sample, the best target match, the second signal sample and the best non-target match of the currently mentioned embodiment of the present invention.

Furthermore, it will be clear that by using a set of reference target signal segments that is based on the plurality of reference target signal segments and a set of reference non-target signal segments that is based on the plurality of reference non-target signal segments the voting process can eliminate one of the two classifications without having to use all reference target signal segments of the plurality of reference target signal segments and all reference non-target signal segments of the plurality of reference non-target signal segments, respectively. As a result, the voting process can be performed faster.

In an embodiment of the system according to the invention, the processor is configured and arranged to remove the classification of the detected first signal segment or the classification of the detected second signal segment that based on the voting process is incorrect. The person skilled in the art will appreciate that the voting process results in a so-called winner, i.e. either the classification of the detected first signal segment as being a target signal segment or the classification of the detected second signal segment as being a non-target signal segment is correct. The classification that in accordance with the voting process is to be regarded as incorrect will be removed.

In an embodiment of the system according to the invention, the processor is configured and arranged to apply a predetermined detection boundary that is determined based on the target parameter set and/or the non-target parameter set, the detection boundary allowing a classification of detected signal segments as target signal segments or as non-target signal segments. The detection boundary can for example be a boundary plane in the feature space of wavelet coefficients that is determined by training on a labeled training set of target signal segments and non-target signal segments. In this way, another way to make a distinction between target signal segments and non-target signal segments of a single-channel EEG-recording can be provided. Hence, false positive detections or incorrect classifications for that matter can be reduced.

In an embodiment of the system according to the invention, the processor is configured and arranged to determine an optimized target parameter set that comprises wavelet coefficients that are indicative specifically for the plurality of reference target signal segments and/or an optimized non-target parameter set that comprises wavelet coefficients that are indicative specifically for the plurality of reference non-target signal segments. Determination of the optimized target parameter set and the optimized non-target parameter set can be achieved in several ways. A first way of doing this involves comparing of the wavelet coefficients of the target parameter set and the wavelet coefficients of the non-target parameter set, wherein wavelet coefficients that occur both in the target parameter set and in the non-target parameter set are removed from the target parameter set and/or from the non-target parameter set. In this way, overlap between the target parameter set and the non-target parameter set can be reduced.

A second way of determining the optimized target parameter set and the optimized non-target parameter set involves using the resulting sensitivity and specificity of a system, e.g. a classifier unit, that is adapted to classify a detected first signal segment as being a target signal segment and a detected second signal segment as being a non-target signal segment.

As a result of any one of the above-mentioned first way and second way, the thus optimized target parameter set and non-target parameter set enable an improved distinction between target signal segments and non-target signal segments of a single-channel EEG-recording. Hence, false positive detections or incorrect classifications for that matter can be reduced.

In an embodiment of the system according to the invention, the processor is configured and arranged to apply a predetermined detection boundary that is determined based on the optimized target parameter set and/or the optimized non-target parameter set, the detection boundary allowing an improved classification of detected signal segments as target signal segments or as non-target signal segments. In this way, an improved distinction between target signal segments and non-target signal segments of a single-channel EEG-recording can be achieved. Hence, false positive detections or incorrect classifications for that matter can be reduced.

In an embodiment of the system according to the invention, the system further comprises a data storage unit that is operatively connected to the processor, wherein the data storage unit is configured and arranged to store at least one of the single-channel EEG-recording, the signal obtained from the single-channel EEG-recording, and a classification of a detected signal segment of said signal as a target signal segment or as a non-target signal segment as a result of the method performed by the processor.

In an embodiment of the system according to the invention, the system is configured and arranged to be connectable with two electrodes that are arrangeable on a subject's scalp and are configured to record the single-channel EEG-recording and transfer the single-channel EEG-recording to the data storage unit. The person skilled in the art will appreciate that a system that is connected with more than two electrodes, for example three or four electrodes or any other suitable number, to record the single-channel EEG-recordings also falls within the scope of the present invention as such system is also connected with two electrodes as defined by this exemplary embodiment of the system according to the invention.

### BRIEF DESCRIPTION OF THE DRAWINGS

Further features and advantages of the invention will become apparent from the description of the invention by way of exemplary and non-limiting embodiments of a method according to the present invention and a device and a system for performing the method according to the invention.

The person skilled in the art will appreciate that the described embodiments of the method according to the present invention and the device and the system for performing the method according to the invention are exemplary in nature only and not to be construed as limiting the scope of protection in any way. The person skilled in the art will realize that alternatives and equivalent embodiments of the method according to the present invention and the device and the system for performing the method according to the invention can be conceived and reduced to practice without departing from the scope of protection of the present invention.

Reference will be made to the figures on the accompanying drawing sheets. The figures are schematic in nature and therefore not necessarily drawn to scale. Furthermore, equal reference numerals denote equal or similar parts. On the attached drawing sheets,

FIG. 1 shows how a target signal segment of a test signal that is obtained from a single-channel EEG-recording can be classified as a target signal segment or as a non-target signal segment using the method according to the invention. For the step of determining a temporal proximity of the first time stamp t1 that is assigned to a detected first signal segment of the test signal and the second time stamp t2 that is assigned to a detected second signal segment of the test signal, two exemplary ways of doing this are provided, and

FIG. 2 shows a schematic layout of exemplary, non-limiting embodiments of a device and a system according to the invention.

### DETAILED DESCRIPTION OF THE INVENTION

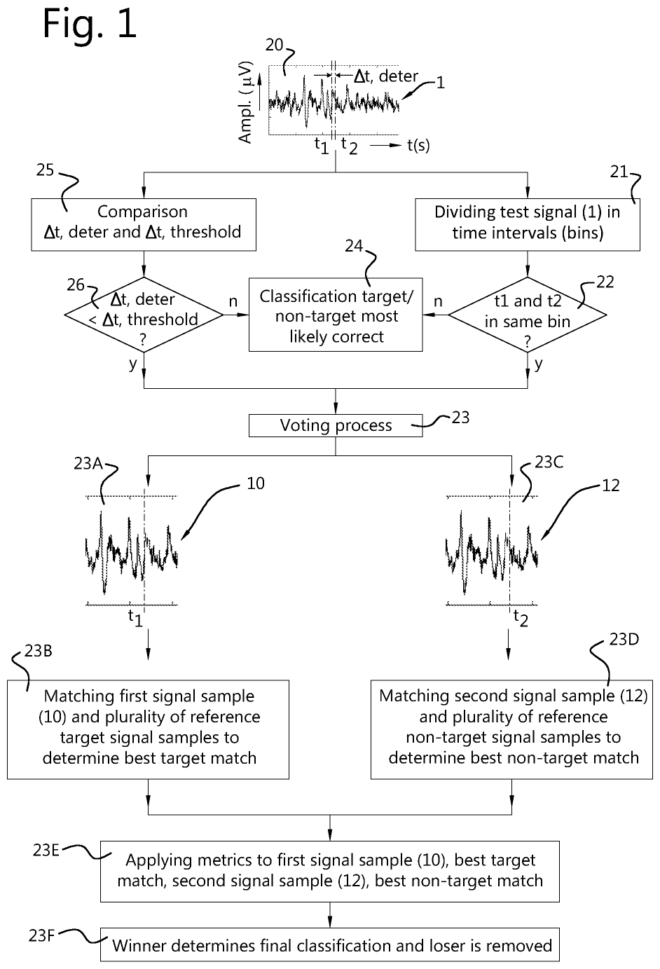

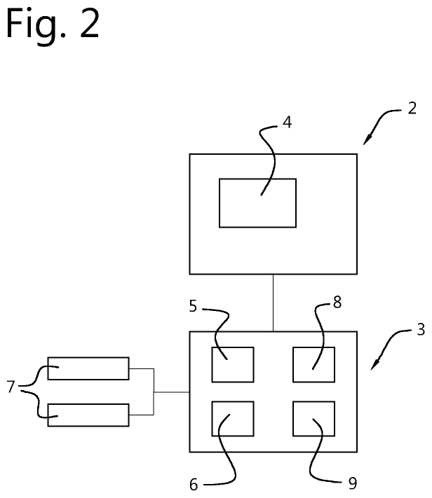

FIG. 1 shows how a target signal segment of a test signal 1 that is obtained from a single-channel EEG-recording can be classified as a target signal segment or as a non-target signal segment using the method according to the invention. The test signal 1 that is schematically represented in FIG. 1, can be obtained from a single-channel EEG-recording. The detected test signal 1 can be presented to a device 2 and a system 3 according to the invention. FIG. 2 shows a schematic layout of exemplary, non-limiting embodiments of the device 2 and the system 3 according to the invention.

In a first step 20 of the method according to the invention, the test signal 1 shown in FIG. 1 can be obtained from a single-channel EEG-recording. Single-channel EEG-recordings can be acquired in various known ways including for example using a differential electrode pair, using a single electrode in combination with a reference (REF) electrode or a ground (GND) electrode, or using a differential electrode pair in combination with a REF electrode or a GND electrode. In accordance with the latter exemplary electrode configuration, the individual electrodes of the differential electrode pair can be positioned at specific midline frontal, e.g. Fz, midline vertex, e.g. Cz, midline parietal, e.g. Pz, left medial temporal, e.g. T3, right medial temporal, e.g. T4, positions on a patient's scalp in accordance with an extended 10-20 EEG system with the REF electrode positioned, for example, on an ear of the patient.

The person skilled in the art will appreciate that in the same way single-channel EEG-recordings can be used to obtain a plurality of reference target signal segments and a plurality of reference non-target signal segments.

It is noted that in the context of the present invention target signal segments of a single-channel EEG-recording are to be construed as signal segments that are indicative for a patient being delirious or suffering from related encephalopathy, whereas non-target signal segments of a single-channel EEG-recording are to be construed as signal segments that are indicative for artifacts such as for example eye artifacts, artifacts related to muscle activity, or artifacts related to a combination of such artifacts.

The reference target signal samples of the plurality of reference target signal samples and the reference non-target signal samples of the plurality of reference non-target signal samples can have a predefined duration of for example 15 minutes. However, any suitable predefined duration can be used as long as the acquired single-channel EEG-recordings enable obtaining suitable reference target signal segments and reference non-target signal segments.

The reference target signal segments of the plurality of reference target signal segments can be mutually different. The same holds for the reference non-target signal segments of the plurality of non-target signal segments. The plurality of reference target signal segments can for example comprise more than 1000 reference target signal segments. The same holds for the plurality of non-target signal segments.

In accordance with the method of the present invention a target parameter set that is indicative for the plurality of reference target signal segments is applied to the test signal 1 to detect a first signal segment and to classify the detected first signal segment as a target signal segment. In the present example the target parameter set comprises wavelet coefficients that are most representative for the reference target signal segments. The wavelet coefficients have been determined based on the plurality of reference target signal samples using a training process that can involve a machine learning algorithm. The machine learning algorithm can for example use neural networks or deep neural networks.

Upon detecting the first signal segment, a first time stamp t1 is assigned to it.

Next, a non-target parameter set that is indicative for the plurality of reference non-target signal segments is applied to the same test signal 1 to detect a second signal segment and to classify the detected second signal segment as a non-target signal segment. In the present example the non-target parameter set comprises wavelet coefficients that are most representative for the reference non-target signal segments. The wavelet coefficients have been determined based on the plurality of reference non-target signal samples using another training process that can involve another machine learning algorithm that for example can use neural networks or deep neural networks.

Upon detecting the second signal segment, a second time stamp t2 is assigned to it.

In an exemplary embodiment of the method according to the invention, the wavelet coefficients of the target parameter set and the wavelet coefficients of the non-target parameter set can be compared to optimize the target and non-target parameter sets by removing from either one of them wavelet coefficients that occur in both of them. In this way, overlap between the target parameter set and the non-target parameter set can be reduced. Thus, an optimized target parameter set that comprises wavelet coefficients that are indicative specifically for the plurality of reference target signal segments, and an optimized non-target parameter set that comprises wavelet coefficients that are indicative specifically for the plurality of reference non-target signal segments can be obtained. As a result, the optimized target parameter set and the optimized non-target parameter set enable an improved distinction between target signal segments and non-target signal segments of the single-channel EEG-recording. Hence, false positive detections or incorrect classifications for that matter can be reduced.

In a next step of the method according to the present invention, a temporal proximity of the first time stamp t1 and the second time stamp t2 is determined. The person skilled in the art will appreciate that the temporal proximity of the first time stamp t1 and the second time stamp t2 can be determined in several different ways. FIG. 1 shows two exemplary ways of doing this.

A first exemplary way of doing this that is explained in relation to step 21 in FIG. 1, involves dividing the obtained test signal 1 in time intervals of a predefined length. These time intervals can be referred to as bins. Preferably, the time intervals or bins have a predefined equal length. The predefined length of the time intervals can be chosen depending on specific requirements such as desired accuracy. Suitable predefined lengths of the time intervals range between 0.25 s and 3 s. Preferably, the time intervals have a predefined length of 1 s. Determining the temporal proximity of the first time stamp t1 and the second time stamp t2 is based on establishing if the first time stamp t1 and the second time stamp t2 fall within the same time interval or not. This is indicated as step 22 in FIG. 1. If the first time stamp t1 and the second time stamp t2 fall within the same time interval or bin, then the voting process, which is indicated as step 23 in FIG. 1, is required and will be performed to determine whether classification of the detected first signal segment as a target signal segment or classification of the detected second signal segment as a non-target signal segment is correct. If the first time stamp t1 and the second time stamp t2 fall in different time intervals, i.e. the first time stamp t1 and the second time stamp t2 do not fall within the same bin, then the voting process is not required and preferably the voting process is not performed. In this case, the classification of the detected first signal segment as being a target signal segment and the classification of the detected second signal segment as being a non-target signal segment are most likely both correct. This is indicated as step 24 in FIG. 1.

A second way of determining the temporal proximity of the first time stamp t1 and the second time stamp t2 that is explained in relation to step 25 in FIG. 1, involves determining a time difference Δt,deter between the first time stamp t1 and the second time stamp t2. The determined time difference Δt,deter is compared with a predefined threshold Δt,threshold. The person skilled in the art will appreciate that the threshold Δt,threshold is chosen such that when the determined time difference Δt,deter is smaller than the threshold Δt,threshold, it is not likely or not possible at all that the classification of the detected first signal segment is assigned as being a target signal segment and the classification of the detected second signal segment is assigned as being a non-target signal segment can both be correct. Hence, as indicated in step 26 in FIG. 1, when the determined time difference Δt,deter is smaller than the threshold Δt,threshold, then a voting process, which is indicated as step 23 in FIG. 1, will be performed to determine whether classification of the detected first signal segment as a target signal segment or the classification of the detected second signal segment as a non-target signal segment is correct.

However, if the determined time difference Δt,deter is equal to or larger than the threshold Δt,threshold, then the classification of the detected first signal segment as being a target signal segment and the classification of the detected second signal segment as being a non-target signal segment are most likely both correct. This is indicated as step 24 in FIG. 1.

The person skilled in the art will appreciate that any suitable threshold Δt,threshold can be chosen as long as it allows to establish whether the classification of the detected first signal segment as being a target signal segment and/or the classification of the detected second signal segment as being a non-target signal segment can be correct. Suitable values for the predefined threshold range between 0.25 s and 3 s. Preferably, the threshold is 1 s. Based on the above, it will be clear that the voting process eliminates one of the classifications. As a result, the method according to the present invention can reduce false positive detections or incorrect classifications for that matter.

The voting process of the method of the present invention comprises a step 23A of generating a first signal sample 10 that comprises the detected first signal segment to which the first time stamp t1 has been assigned.

In a next step 23B of the voting process the generated first signal sample 10 is matched with the plurality of reference target signal segments to determine a best target match.

In a similar way, another step 23C in the voting process is generating a second signal sample 12 that comprises the detected second signal segment to which the second time stamp t2 has been assigned. Then, in a next step 23D of the voting process, the second signal sample 12 is matched with the plurality of reference non-target signal segments to determine a best non-target match.

In accordance with the present invention, matching the first signal sample 10 with the plurality of reference target signal segments to determine the best target match can involve for example curve fitting in the time domain of the first signal sample 10 with the plurality of reference target signal segments. In an analogous way, matching the second signal sample 12 with the plurality of reference non-target signal segments to determine the best non-target match can involve for example curve fitting in the time domain of the second signal sample 12 with the plurality of reference non-target signal segments. Curve fitting in the time domain may include comparing the signal shape of the first signal sample 10 with the signal shapes of the reference target signal segments of the plurality of reference target signal segments and comparing of the signal shape of the second signal sample 12 with the signal shapes of the reference non-target signal segments of the plurality of reference non-target signal segments. In this case, the curve fit resulting in for example the smallest residue can be chosen to determine the best target match and the best non-target match, respectively. However, other aspects related to the curve fitting process can of course also be regarded to determine the best target match and the best non-target match, respectively.

The person skilled in the art will appreciate that curve fitting in the time domain is just an example of the analysis methods that are available to determine the best target match and the best non-target match, respectively. Examples of analysis methods include for example Fast Fourier Transform (FFT), linear signal analysis techniques involving determination of coherence, non-linear signal analysis techniques involving determination of phase synchronization and/or generalized synchronization, template matching, and parametric models including the use of wavelets.

As a next step 23E of the voting process, metrics are applied to the first signal sample 10, the best target match, the second signal sample 12 and the best non-target match parameter to determine whether the classification of the detected first signal segment as a target signal segment is correct, or whether the classification of the detected second signal segment as a non-target signal segment is correct.

In accordance with the present invention, applying metrics to the first signal sample 10, the best target match, the second signal sample 12 and the best non-target match can be done in several different ways. A first way of doing this is by establishing and comparing a correlation in the time domain. A second way of doing this is by establishing and comparing a goodness of fit in the wavelet domain. By applying either one of these techniques it can be determined whether the classification of the detected first signal segment as being a target signal segment or the classification of the detected second signal segment as being a non-target signal segment is correct.

Based on the above it will be clear that the voting process of the method of the present invention will result in a so-called winner, i.e. the voting process eliminates one of the two classifications and thereby will determine the final classification as target signal segment or as non-target signal segment. As a result, false positive classifications or incorrect detections for that matter can be reduced. The loser is removed. This is indicated as step 23F in FIG. 1.

FIG. 2 shows a schematic layout of exemplary, non-limiting embodiments of a device 2 and a system 3 according to the invention. The device 2 can be construed as a detector that is configured and arranged to be used with the system 3 that is configured and arranged to detect and classify a segment of a signal that is obtained from a single-channel EEG-recording as a target signal segment or as a non-target signal segment. The device 2 has a database 4 that comprises at least one of a plurality of reference target signal segments that are obtained from signals that are obtained from reference single-channel EEG-recordings, a set of reference target signal segments that is based on the plurality of reference target signal segments, a plurality of reference non-target signal segments that are obtained from signals that are obtained from reference single-channel EEG-recordings, a set of reference non-target signal segments that is based on the plurality of reference non-target signal segments, a target parameter set that is indicative for the plurality of reference target signal segments, and a non-target parameter set that is indicative for a plurality of reference non-target signal segments. As mentioned above, the target parameter set and the non-target parameter set can be obtained via respective training processes that may involve respective machine learning algorithms that may use respective deep neural networks.

The device 2 according to the invention enables an improved distinction between target signal segments and non-target signal segments of a signal that is obtained from a single-channel EEG-recording. As a result, false positive detections or incorrect classifications for that matter as discussed above can be reduced.

The system 3 according to the invention is configured and arranged to detect and classify a segment of a signal that is obtained from a single-channel EEG-recording as a target signal segment or as a non-target signal segment in accordance with the method of the present invention. The system 3 comprises a processor 5 that is configured and arranged to perform the method according to the present invention on said signal when being operatively connected to the device 2 according to the present invention.

In this way, the system 3 and the device 2 when being operatively connected can be used to achieve an improved distinction between target signal segments and non-target signal segments of a signal that is obtained from a single-channel EEG-recording. As a result, false positive detections or incorrect classifications for that matter as discussed above can be reduced. The person skilled in the art will appreciate that the device 2 and the system 3 can be implemented as separate units as is schematically shown in FIG. 2. However, the device 2 and the system 3 can also be implemented as an integrated unit (not shown).

The system 3 shown in FIG. 2 further comprises a data storage unit 6 that is operatively connected to the processor 5. The data storage unit 6 can be configured and arranged to store at least one of the single-channel EEG-recording, the signal obtained from the single-channel EEG-recording, and a classification of a detected signal segment of said signal as a target signal segment or as a non-target signal segment as a result of the method performed by the processor 5.

In the exemplary, non-limiting embodiment of the system 3 shown in FIG. 2, the system 3 is connected with two electrodes 7 that are arrangeable on a subject's scalp and are configured to record the single-channel EEG-recordings and transfer the single-channel EEG-recordings to the data storage unit 6. The system 3 can further be configured to comprise application software 8 and a display unit 9, such as a screen.

The present invention can be summarized as relating to a method for detecting and classifying a segment of a signal 1 that is obtained from an EEG-recording as a target signal segment or as a non-target signal segment. The method comprises a voting process to determine whether classification of a first detected segment of the signal as a target signal segment or classification of a second detected segment of the signal as a non-target signal segment is correct. The invention further relates to a device 2 and a system 3 that are configured and arranged to perform the method according to the invention.

It will be clear to a person skilled in the art that the scope of the present invention is not limited to the examples discussed in the foregoing but that several amendments and modifications thereof are possible without deviating from the scope of the present invention as defined by the attached claims. In particular, combinations of specific features of various aspects of the invention may be made. An aspect of the invention may be further advantageously enhanced by adding a feature that was described in relation to another aspect of the invention. While the present invention has been illustrated and described in detail in the figures and the description, such illustration and description are to be considered illustrative or exemplary only, and not restrictive.

The present invention is not limited to the disclosed embodiments. Variations to the disclosed embodiments can be understood and effected by a person skilled in the art in practicing the claimed invention, from a study of the figures, the description and the attached claims. In the claims, the word “comprising” does not exclude other steps or elements, and the indefinite article “a” or “an” does not exclude a plurality. The mere fact that certain measures are recited in mutually different dependent claims does not indicate that a combination of these measures cannot be used to advantage. Any reference numerals in the claims should not be construed as limiting the scope of the present invention.

What is claimed is:

1. A processor implemented data processing method for detecting and classifying a segment of a signal that is obtained from a single-channel EEG-recording as a target signal segment or as a non-target signal segment, the method comprising:
providing a signal that is obtained from a single-channel EEG-recording;
applying to said signal a target parameter set, which target parameter set is indicative for a plurality of reference target signal segments that are obtained from reference single channel EEG-recordings, to detect a first signal segment of said signal and to classify the detected first signal segment as a target signal segment, wherein the target parameter set comprises wavelet coefficients that are determined using wavelet decomposition of the plurality of reference target signal segments;
assigning a first time stamp (t1) to the detected first signal segment;
applying to said signal a non-target parameter set, which non-target parameter set is indicative for a plurality of reference non-target signal segments that are obtained from reference single-channel EEG-recordings, to detect a second signal segment of said signal and to classify the detected second signal segment as a non-target signal segment, wherein the non-target parameter set comprises wavelet coefficients that are determined using wavelet decomposition of the plurality of reference non target signal segments;
assigning a second time stamp (t2) to the detected second signal segment;
determining a time difference between the first time stamp (t1) and the second time stamp (t2);
when said time difference is smaller than a predetermined threshold, determining that a voting process is required to determine whether classification of the detected first signal segment as a target signal segment or classification of the detected second signal segment as a non-target signal segment is correct; and
upon establishing that said voting process is required, performing said voting process.

2. The data processing method according to claim 1, wherein, in the step of applying to said signal the target parameter set, the detected first signal segment is classified as a target signal segment when the detected first signal segment is indicative for a patient being delirious or suffering from related encephalopathy, and
wherein, in the step of applying to said signal the non-target parameter set, the detected second signal segment is classified as a non-target signal segment when the selected second signal segment is indicative for artifacts.

3. The data processing method according to claim 1, wherein performing the voting process comprises:
generating a first signal sample that comprises the detected first signal segment;
matching the first signal sample with the plurality of reference target signal segments to determine a best target match;
generating a second signal sample that comprises the detected second signal segment;
matching the second signal sample with the plurality of reference non-target signal segments to determine a best non-target match; applying metrics to the first signal sample, the best target match, the second signal sample and the best non-target match to determine:
whether the classification of the detected first signal segment as a target signal segment is correct; or
whether the classification of the detected second signal segment as a non-target signal segment is correct.

4. The data processing method according to claim 1, wherein performing the voting process comprises:
generating a first signal sample that comprises the detected first signal segment;
matching the first signal sample with a set of reference target signal segments that is based on the plurality of reference target signal segments to determine a best target match;
generating a second signal sample that comprises the detected second signal segment;
matching the second signal sample with a set of reference non-target signal segments that is based on the plurality of reference non-target signal segments to determine a best non-target match;
applying metrics to the first signal sample, the best target match, the second signal sample and the best non-target match to determine:
whether the classification of the detected first signal segment as a target signal segment is correct; or
whether the classification of the detected second signal segment as a non target signal segment is correct.

5. The data processing method according to claim 1, further comprises removing the classification of the detected first signal segment or the classification of the detected second signal segment that based on the voting process is incorrect.

6. The data processing method according to claim 1, wherein a predetermined detection boundary, which is determined based on the target parameter set and/or the non-target parameter set, is applied that allows classification of detected signal segments as target signal segments or as non-target signal segments.

7. The data processing method according to claim 1, further comprises determining an optimized target parameter set that comprises wavelet coefficients that are indicative specifically for the plurality of reference target signal segments and/or an optimized non-target parameter set that comprises wavelet coefficients that are indicative specifically for the plurality of reference non-target signal segments.

8. The data processing method according to claim 7, wherein based on the optimized target parameter set and/or the optimized non-target parameter set a detection boundary is determined that allows improved classification of detected signal segments as target signal segments or as non-target signal segments.

9. (canceled)

10. A system that is configured and arranged to detect and classify a segment of a signal that is obtained from a single-channel EEG-recording as a target signal segment or as a non-target signal segment, the system comprising a processor that is configured and arranged to perform on said signal when being operatively connected to a device having a database, the process steps of:
providing a signal that is obtained from a single-channel EEG-recording;
applying to said signal a target parameter set, which target parameter set is indicative for a plurality of reference target signal segments that are obtained from reference single channel EEG-recordings, to detect a first signal segment of said signal and to classify the detected first signal segment as a target signal segment, wherein the target parameter set comprises wavelet coefficients that are determined using wavelet decomposition of the plurality of reference target signal segments;
assigning a first time stamp (t1) to the detected first signal segment;
applying to said signal a non-target parameter set, which non-target parameter set is indicative for a plurality of reference non-target signal segments that are obtained from reference single-channel EEG-recordings, to detect a second signal segment of said signal and to classify the detected second signal segment as a non-target signal segment, wherein the non-target parameter set comprises wavelet coefficients that are determined using wavelet decomposition of the plurality of reference non target signal segments;
assigning a second time stamp (t2) to the detected second signal segment;
determining a time difference between the first time stamp (t1) and the second time stamp (t2);
when said determined time difference is smaller than a predetermined threshold, determining if a voting process is required to determine whether classification of the detected first signal segment as a target signal segment or classification of the detected second signal segment as a non-target signal segment is correct; and
upon establishing that said voting process is required, performing said voting process;
wherein the database comprises at least one of:
a plurality of reference target signal segments that are obtained from reference single-channel EEG-recordings;
a set of reference target signal segments that is based on the plurality of reference target signal segments;
a plurality of reference non-target signal segments that are obtained from reference single-channel EEG-recordings;
a set of reference non-target signal segments that is based on the plurality of reference non-target signal segments;
a target parameter set that is indicative for the plurality of reference target signal segments, wherein the target parameter set comprises wavelet coefficients that are determined using wavelet decomposition of the plurality of reference target signal segments; and
a non-target parameter set that is indicative for a plurality of reference non target signal segments, wherein the non-target parameter set comprises wavelet coefficients that are determined using wavelet decomposition of the plurality of reference non-target signal segments.

11. (canceled)

12. The system according to claim 10, wherein the processor is configured and arranged to perform the voting process comprising the process steps of:
generating a first signal sample that comprises the detected first signal segment;
matching the first signal sample with the plurality of reference target signal segments to determine a best target match;
generating a second signal sample that comprises the detected second signal segment;
matching the second signal sample with the plurality of reference non-target signal segments to determine a best non-target match;
applying metrics to the first signal sample, the best target match, the second signal sample and the best non-target match to determine:
whether the classification of the detected first signal segment as a target signal segment is correct; or
whether the classification of the detected second signal segment as a non target signal segment is correct.

13. The system according to claim 10, wherein the processor is configured and arranged to perform the voting process comprising the process steps of:
generating a first signal sample that comprises the detected first signal segment;
matching the first signal sample with a set of reference target signal segments that is based on the plurality of reference target signal segments to determine a best target match;
generating a second signal sample that comprises the detected second signal segment;
matching the second signal sample with a set of reference non-target signal segments that is based on the plurality of reference non-target signal segments to determine a best non-target match;
applying metrics to the first signal sample, the best target match, the second signal sample and the best non-target match to determine:
whether the classification of the detected first signal segment as a target signal segment is correct; or
whether the classification of the detected second signal segment as a non target signal segment is correct.

14. The system according to claim 10, wherein the processor is configured and arranged to remove the classification of the detected first signal segment or the classification of the detected second signal segment that based on the voting process is incorrect.

15. The system according to claim 10, wherein the processor is configured and arranged to apply a predetermined detection boundary that is determined based on the target parameter set and/or the non-target parameter set, the detection boundary allowing a classification of detected signal segments as target signal segments or as non-target signal segments.

16. The system according to claim 10, wherein the processor is configured and arranged to determine an optimized target parameter set that comprises wavelet coefficients that are indicative specifically for the plurality of reference target signal segments and/or an optimized non-target parameter set that comprises wavelet coefficients that are indicative specifically for the plurality of reference non-target signal segments.

17. The system according to claim 16, wherein the processor is configured and arranged to apply a predetermined detection boundary that is determined based on the optimized target parameter set and/or the optimized non-target parameter set, the detection boundary allowing an improved classification of detected signal segments as target signal segments or as non-target signal segments.

18. The system according to claim 10, further comprising a data storage unit that is operatively connected to the processor, wherein the data storage unit is configured and arranged to store at least one of the single-channel EEG-recording and the signal obtained from the single-channel EEG-recording, and a classification of a detected signal segment of said signal as a target signal segment or as a non-target signal segment as a result of the method performed by the processor.

19. The system according to claim 18, wherein the system is configured and arranged to be connectable with two electrodes that are arrangeable on a subject's scalp and are configured to record the single-channel EEG-recording and transfer the single-channel EEG-recording to the data storage unit.
